(19) United States
(12) Patent Application Publication
(10) Pub. No.: US 2004/0122242 A1
(43) Pub. Date: Jun. 24, 2004

(54) Ruthenium-molybdenum catalyst for hydrogenation in aqueous solution

(76) Inventor: Daniel Campos, Lancaster, PA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/328,130

(22) Filed: Dec. 23, 2002

(51) Int. Cl. C 07D 3 7/02

(52) U.S. Cl. 549/429; 568/861

(57) ABSTRACT

An improved catalyst of ruthenium, molybdenum and, optionally, tin with an inert support used for hydrogenation of an hydrogenatable precursor in an aqueous solution and a method for using the catalyst in the production of tetrahydrofuran and 1,4-butanediol from such a hydrogenatable precursor in an aqueous solution.

### BACKGROUND OF THE INVENTION

1. Field of the Invention

This invention relates to a metallic catalyst with an inert support for hydrogenation in an aqueous solution and a method for using the catalyst in the production of tetrahydrofuran and 1,4-butanediol from a hydrogenatable precursor in an aqueous solution.

2. Description of the Related Art

Various methods and reaction systems have been proposed in the past for manufacturing tetrahydrofuran (THF) and 1,4 butanediol (BDO) by catalytic hydrogenation of gamma butyrolactone (GBL), maleic acid (MAC), maleic anhydride (MAN), succinic acid (SAC) and/or related hydrogenatable precursors. Also, a variety of hydrogenation catalysts have been historically proposed for this purpose, including various transition metals and their combinations deposited on various inert supports. Many of these catalysts are proposed for use in hydrogenations carried out in an organic solvent or organic reaction media and not in an aqueous solution phase. In fact, at least one prior publication suggests that water and succinic acid may be considered as inhibitors to the desired catalysis, see Bulletin of Japan Petroleum Institute, Volume 12, pages 89 to 96 (1970).

U.S. Pat. No. 4,973,717 discloses a process for producing tetrahydrofuran and 1,4-butanediol by hydrogenation of gamma-butyrolactone using a catalyst comprising a noble metal of Group VIII (which includes among others Pd and Ru) alloyed with at least one metal capable of alloying the noble metal. Preferably, a second component of Re, W or Mo is added to the alloyed noble metal. The process solvent is water or an inert organic solvent such as dioxane.

U.S. Pat. No. 5,478,952, incorporated herein by way of reference, discloses a catalyst for aqueous phase hydrogenations. This catalyst consists of Ru and Re wherein both metal components are present in a highly dispersed reduced state on a carbon support which is characterized by a BET surface area of less than 2,000 m²/g.

U.S. Pat. No. 6,008,384 discloses a catalyst of highly dispersed, reduced Ru and Re in the presence of Sn on a carbon support used for an improved hydrogenation process for the production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen. This patent is incorporated herein by way of reference.

U.S. Pat. No. 5,698,749 discloses a process for producing 1,4-butanediol by aqueous hydrogenation of a hydrogenatable precursor using a catalyst comprised of a noble metal of Group VIII (which includes among others Pd and Ru) and at least one of Re, W and Mo on a carbon support pretreated with an oxidizing agent.

### SUMMARY OF THE INVENTION

The invention is a hydrogenation catalyst comprising about 0.5% to 15% of ruthenium, about 0.1% to 5% molybdenum and, optionally, tin with an inert catalyst support, where the percentages are relative to the total weight of support and catalyst, and where the weight ratio of ruthenium to molybdenum is between 2.5 and 4.0.

A method for making tetrahydrofuran, 1,4-butanediol or mixtures thereof by hydrogenating a hydrogenatable precursor in a reactor in the presence of a hydrogenation catalyst comprising about 0.5% to 15% of ruthenium, about 0.1% to 5% molybdenum, and, optionally, tin with an inert catalyst support, where the percentages are relative to the total weight of support and catalyst, and where the weight ratio of ruthenium to molybdenum is between 2.5 and 4.0 and recovering at least one hydrogenatable product from the reactor.

### DETAILED DESCRIPTION OF THE INVENTION

This invention is a bimetallic Ru—Mo (ruthenium-molybdenum) catalyst and a trimetallic Ru—Mo—Sn (ruthenium-molybdenum-tin) catalyst that exhibits certain advantages when employed during hydrogenation of a hydrogenatable precursor in an aqueous solution. The invention also provides an improved process or method for making tetrahydrofuran, 1,4-butanediol or mixtures thereof by hydrogenating a hydrogenatable precursor such as gamma butyrolactone, maleic anhydride, maleic acid, succinic acid, or mixtures thereof. As such, the catalysts of this invention and the process of using these catalysts may be viewed as an improvement of the bimetallic Ru—Re (ruthenium-rhenium) carbon-supported catalyst of U.S. Pat. No. 5,478,952 and of the trimetallic Ru—Re—Sn carbon-supported catalyst of U.S. Pat. No. 6,008,384.

It has been discovered that the addition of specified amounts of molybdenum to a ruthenium catalyst increases the activity and selectivity for products such as tetrahydrofuran and 1,4-butanediol. In addition to comparing favorably with the results obtained with the above referenced Ru—Re catalysts, the inventive Ru—Mo catalyst has the further advantage of substituting molybdenum, a lower cost and more available metal, for rhenium, an expensive metal with a very limited world supply. It has been additionally discovered that the addition of tin to this ruthenium-molybdenum catalyst leads to a further improved control of selectivity among the more useful products, such as tetrahydrofuran and 1,4-butanediol, concurrently with reduced relative production of undesirable by-products such as n-butanol, n-propanol and volatile hydrocarbons such as methane, ethane, propane and butane. Although not confining possible explanation for this discovery to any single rationale or theory, it is currently believed that the addition of relatively small amounts of tin moderates the high catalytic activity of the ruthenium-molybdenum catalyst and the overall rate of hydrogenation so as to improve selectivity to the desired products.

The improved bimetallic hydrogenation catalyst of this invention contains about 0.5% to 15% by weight of Ru, about 0.1% to 5% by weight of Mo, with a weight ratio of Ru to Mo of between 2.5 and 4.0. The improved trimetallic hydrogenation catalyst of this invention contains about 0.5% to 15% by weight of Ru, about 0.1% to 5% by weight of Mo and about 0.1% to 4% by weight of Sn. Additionally, the trimetallic catalyst can have a weight ratio of Ru to Mo of between 2.5 and 4.0. Both catalysts are used with an inert support and the percentages are relative to the total weight of the support plus the catalyst. Preferably, both the bimetallic and the trimetallic catalysts have about 0.8% to 6% of Ru and about 0.1% to 2.5% Mo. Preferably, the trimetallic catalyst has about 0.1% to 2.0% Sn. The inert support can be carbon, TiO₂ or some other inert material.

The hydrogenation catalyst according to the present invention involves both the ruthenium and molybdenum being present with an inert support, optionally with an effective amount of tin. As suggested herein, the presence of the tin is presently viewed as moderating the high catalytic activity of the bimetallic Ru—Mo system to afford improved control of selectivity during hydrogenation at commercial scale operation. This results in a superior yield of desired products and control of the ratio of tetrahydrofuran to by-products being produced without significantly promoting over-hydrogenation and production of undesirable by-products. Consistent with this view, the respective lower limit or minimum loading of ruthenium and molybdenum metals relative to the inert support is somewhat higher than it would be for the bimetallic catalyst without tin in order to at least partially compensate for the presence of tin. As noted above, the upper limit of the ruthenium and molybdenum metal will be about 15% ruthenium and about 5% molybdenum on the same basis. However, it should be appreciated that although concentrations of ruthenium and molybdenum above these upper limits may be operative and as such should be considered equivalent for purposes of the present invention, but such concentrations are believed to offer little advantage in terms of convenience and/or cost.

The carbon useful as a catalyst support in the present invention is preferably a porous particulate solid characterized by a size distribution typically ranging from about 5 to 100 micrometers for slurry applications and from about 0.8 to 4 mm for fixed bed applications and a BET surface area typically ranging from a few hundred to nearly 2,000 m²/g. Preferably, the carbon support material will be commercially available material having an average particle size of about micrometers for slurry applications and about 3 mm for fixed bed applications and a BET surface area from about 700 to about 1,600 m²/g. The catalyst support can be manufactured to have a latent acid, a neutral or a basic pH. Optionally, the catalyst support can be treated prior to metal deposition by one or more techniques as generally known in the art, such as impregnation with alkali metal salts and/or calcination or acid wash. Examples of suitable carbon supports are SX-2 and Darco KBB carbons, supplied by Norit Americas Inc., with BET surface areas of 700 and 1,500 m²/g, respectively.

Other inert materials useful as catalyst support include titania, silica, alumina, zirconia, silicon carbide, etc. A preferred example of suitable inert support is a titania, such as, Degussa P25 TiO₂ powder. Additionally, the inert support useful in the current invention can be any other inert material as commonly known and commercially available for use in this art.

The actual method of preparing the catalyst according to the present invention can be generally any suitable process as known in the art, provided that the aforementioned composition of metals and inert support is achieved.

One such method is to prepare a water solution of a soluble ruthenium compound, a soluble molybdenum compound or a soluble tin compound, and then add this solution to the inert support. The method of adding the solution to the support can be any technique generally known in the art including by way of example, but not by way of limitation: immersion, spraying, incipient wetness, or the like. The water is evaporated thus depositing the ruthenium, molybdenum or tin compounds on the inert support. The dry or partially dried composite material is then added to water to form an aqueous slurry, and the slurry is then subjected to a reducing atmosphere at an elevated temperature (about 150 to 270° C.) for a time sufficient to reduce the ruthenium, molybdenum and tin. The aqueous catalyst slurry can then be added to the reaction zone for use as a catalyst. Alternatively, the aqueous catalyst slurry can be dried or partially dried and then used as catalyst. Optionally, after the deposition step, the dry or partially dried composite material can be subjected to a reducing atmosphere at the aforementioned elevated temperature while in a solid state, and then used as the catalyst.

A second method related to the above is to perform the process entirely in the presence of water or the aqueous solution of the hydrogenatable precursor. In this technique the water solutions of the ruthenium, molybdenum or tin compounds are commingled with the inert support while subjected to a reducing atmosphere at an elevated temperature (about 150 to 270° C.). This methodology is of particular value and commercial interest in that the catalyst drying steps are eliminated, and that the co-depositing and co-reduction can be literally performed in situ in the hydrogenation reactor and even can be accomplished in the presence of reactants such as maleic acid, succinic acid and/or gamma butyrolactone.

A third method of producing the catalyst is to sequentially deposit, dry and reduce the ruthenium and molybdenum on the inert support, then add the solution of the tin compound, as applicable, and deposit, dry and reduce it at an elevated temperature (about 150 to 270° C.) on the same support. Either or both reduction steps are performed in a reducing atmosphere and at the aforementioned elevated temperature and may be performed dry or in an aqueous slurry. Preferentially, both reduction steps are performed as an aqueous slurry.

It should be further appreciated that various other methods or alternate modes of depositing the ruthenium, molybdenum or tin compounds on the inert support are contemplated as being equivalent methodologies for use in preparing the catalysts according to the present invention. This would include methods such as selective precipitation and the like optionally with or without solvent washing to selectively remove less desirable companion anions and the simultaneous or sequential deposition of the individual metal components all as generally known in the art.

The various metallic compounds useful in the present invention for preparing the catalyst can be generally any such compound that is either water soluble or partially water soluble or can be readily converted to a water soluble or partially water soluble compound that can be deposited on the inert support. This would also include by way of example, but not by way of limitation, such ruthenium compounds as RuCl₃.xH₂O, Ru(NO)(NO₃)₃ and the like. This would include by way of example, but not by way of limitation, such molybdenum compounds as (NH₄)₂MoO₄ and the like. This would further include by way of example, but not by way of limitation, such compounds as K₂SnO₃, Na₂SnO₃, SnCl₄, SnCl₂, Sn(NO₃)₂, SnC₂O₄ and the like. Typically, Na₂SnO₃ or SnCl₄ are, used because of availability and cost.

The reducing agent used for the above catalyst reduction step can generally be any reductant or reducing environment consistent with either liquid phase reduction or vapor phase reduction including by way of example, but not by way of limitation: formaldehyde, hydrazine hydrate, hydroxylamine, sodium hypophosphite, sodium formate, glucose, acetaldehyde, sodium borohydride, hydrogen and the like. When a vapor phase reduction is employed involving gaseous hydrogen with or without an inert diluent gas, such as, nitrogen in the presence of the catalyst precursor, typically the vapor phase reduction is performed at a temperature range of 100 to 500° C., preferably 250 to 300° C. and at atmospheric pressure or up to a pressure of 3000 psig (2.07×10⁷ Pa gage).

The present invention is also the use of either the bimetallic or trimetallic composition for the catalytic hydrogenation of a hydrogenatable precursor in an aqueous solution comprising the steps of:

(a) hydrogenating a hydrogenatable precursor in an aqueous solution in the presence of hydrogen and a catalyst of the above composition, and,

(b) recovering at least one hydrogenated product.

Typically, in the above process, the hydrogenatable precursor is selected from the group consisting of maleic acid, maleic anhydride, fumaric acid, succinic acid, the esters corresponding to these acids, gamma butyrolactone, and mixtures thereof. Typically, the preferred temperature for the hydrogenation step is from 150 to about 260° C. It has been found that at lower temperatures (e.g., 200° C. or lower) BDO is predominantly produced over THF. Conversely, higher temperatures favor the production of THF over BDO. In addition to temperature, the mode of product removal from the reactor is also a critical factor for producing predominantly either THF or BDO. Specifically, removing the product in the vapor phase favors the production of THF over BDO. Conversely, removing the product in the liquid phase favors the production of BDO over THF.

The catalyst are then used for the hydrogenation of a hydrogenatable precursor to tetrahydrofuran and/or 1,4-butanediol. For purposes of the present invention, a hydrogenatable precursor can be, broadly, any compound or material that can be chemically reduced by hydrogenation or hydrogen uptake to yield the desired products. This would include, in particular but again not by way of limitation, various organic compounds containing unsaturation or oxygenated functional groups or both. Most particularly, the aqueous phase catalytic reduction of maleic acid to gamma butyrolactone, 1,4-butanediol and tetrahydrofuran is illustrative of the utility of the method according to the present invention. In this regard, and as illustrated in the examples, it should be appreciated that various products of the sequential hydrogenation reaction are also potential hydrogenatable precursors. That is, in the conversion of maleic acid to tetrahydrofuran the chemical reduction is known to be sequential, involving the rapid addition of hydrogen across the double bond, thereby converting maleic acid to succinic acid. This is followed by the slower addition of hydrogen forming potential intermediates such as gamma butyrolactone and/or 1,4-butanediol and ultimately tetrahydrofuran (corresponding to the uptake of 5 moles of H₂ and production of three moles of H₂O per mole of THF). In commercial production, the overall selectivity to THF production can be significantly influenced by optimizing reaction conditions including maintaining adequate acidity to favor ring closure and cyclic ether production at the expense of diol production, continuous vapor removal of the more volatile products, and subsequent separation and recycle of the lactone. In these cases, the gamma butyrolactone can be viewed as either a co-product or as a recycled hydrogenatable precursor reactant.

The method of using the metallic catalysts to hydrogenate a hydrogenatable precursor according to the present invention can be performed by various modes of operation as generally known in the art. Thus, the overall hydrogenation process can be by use of a fixed bed reactor, various types of agitated slurry reactors, either gas or mechanically agitated or the like, operated in either a batch or continuous mode, wherein an aqueous liquid phase containing the hydrogenatable precursor is in contact with a gaseous phase containing hydrogen at elevated pressure and the particulate solid catalyst. Typically, such hydrogenation reactions are performed at temperatures from about 100° C. to about 300° C. in sealed reactors maintained at pressures from about 1000 to about 3000 psig (7×10⁶ to about 21×10⁶ Pa gage).

When the metallic catalysts of the present invention are used to produce 1,4-butanediol and tetrahydrofuran at a desired or controlled molar ratio, the hydrogenation is preferably performed at a temperature above about 150° C. and below about 260° C. To obtain a high 1,4-butanediol to tetrahydrofuran (BDO/THF) molar ratio, the hydrogenation to those desired products should advantageously be performed at or near the lower end of this temperature range. The method and conditions as the mode of operation will also influence advantageously the BDO/THF molar ratio during hydrogenation. For example, the liquid phase removal of products from the hydrogenation reactor will tend to enhance and maximize 1,4-butanediol production rather than tetrahydrofuran. In contrast, continuous vapor removal of product from the hydrogenation reactor will tend to maximize tetrahydrofuran production at the expense of 1,4-butanediol. Thus, as a practical consideration, low temperature liquid product removal intended to optimize 1,4-butanediol production favors the use of fixed bed catalytic reactors. On the other hand, high temperature vapor phase product removal intended to optimize tetrahydrofuran production favors the use of a slurry or stirred reactor.

The following examples are presented to more fully demonstrate and further illustrate various individual aspects and features of the present invention while the comparative examples are intended to further illustrate the differences and advantages of the present invention. As such, the examples are meant to illustrate the invention, but are not meant to be limiting in any way.

### EXAMPLES

The examples given below measure the relative performance of different catalyst compositions. For comparison purposes, in each of these tests the catalyst metals, the inert support, and the reactants were mixed together in an aqueous system, and the hydrogenation reaction carried out using a fixed procedure. It is understood that alternate procedures for preparing the catalyst and carrying out the hydrogenation reaction may also be used, as described previously. Because a single reaction temperature was chosen for comparison purposes, and because the chosen temperature (250° C.) was toward the high end of the previously described preferred range (200 to 260° C.), the proportion of THF relative to BDO was favored in all these examples. For most of the following examples, about 70% to 85% of the desired two products was THF, with BDO as the remainder. The development of alternate procedures for a particular hydrogenatable precursor and to obtain a particularly desired product composition ratio will be apparent to one skilled in the art and need not involve extensive experimentation.

### Example 1

To a 300-cc autoclave was added 0.4 g of Degussa P25 TiO₂ powder, 0.03 g of RuCl₃.xH₂O and 0.005 g of MoO₃, for an overall composition of 2.5 wt % Ru and 0.83 wt % Mo. Then, 125 g of 20% aqueous gamma butyrolactone (GBL) was added. The autoclave was heated to 250° C. and then pressurized to 2000 psig with H₂ while stirring. The conditions were maintained for 45 minutes, after which it was rapidly cooled down. The products were analyzed by gas chromatography to determine the net molar production rate (STY) and selectivity. The STY was 63.6 mol/Kg of catalyst-hour, where mols=the sum of 1,4-butanediol (BDO) and tetrahydrofuran (THF). The selectivity was 0.56, measured by dividing the sum of the (BDO+THF) STY by the sum of (BDO+THF+byproducts) STY. In terms of the two desired products only, the molar proportion of THF was 87% and the BDO was 13%. This trial is called Example 1a. A repeat scouting test (Example 1b) gave an STY of 35.7 and a selectivity of 0.61. The reason for the lower STY was not determined. A third trial (Example 1 c) essentially confirmed the first set of results, with an STY of 58.1, a selectivity of 0.64, and a proportion for the two desired products of 82% THF and 18% BDO.

### Examples 2-10

The scouting tests described in Example 1a were repeated except for changing the amount of Ru and Mo added. The results for Examples 1 through 10, including any duplicate tests and comparative examples with no added Mo, are summarized in Table 1 below.

### Comparative Example A

The test described in Example 1 was repeated except for omitting the molybdenum. The first trial is called Comparative Example A and the second Comparative Example B.

TABLE 1
Ru—Mo Catalysts on TiO₂ Support

| Example | Wt % Ru | Wt % Mo | STY | Selectivity |
|---|---|---|---|---|
| Comparative A | 2.50 | 0.00 | 31.9 | 0.39 |
| Comparative B | 2.50 | 0.00 | 40.4 | 0.37 |
| Example 1 a | 2.50 | 0.83 | 63.6 | 0.56 |
| Example 1 b | 2.50 | 0.83 | 35.7 | 0.61 |
| Example 1 c | 2.50 | 0.83 | 58.1 | 0.64 |
| Example 2 | 2.50 | 1.67 | 36.6 | 0.77 |
| Example 3 | 2.50 | 2.50 | 19.5 | 0.75 |
| Example 4 | 2.50 | 3.33 | 18.7 | 0.79 |
| Example 5 | 4.00 | 1.33 | 44.9 | 0.64 |
| Example 6 | 5.00 | 0.83 | 32.7 | 0.56 |
| Example 7 | 5.00 | 1.17 | 37.6 | 0.55 |
| Example 8 | 5.00 | 1.67 | 32.1 | 0.70 |
| Example 9 | 5.00 | 2.17 | 32.9 | 0.69 |
| Example 10 | 5.00 | 2.50 | 27.9 | 0.72 |

The results above show that Mo increases the activity of the Ru catalyst on a TiO₂ support when present in relatively small amounts compared to the Ru. An increase in selectivity can be observed over about the same range. The optimum Ru/Mo weight ratio ranges between 2.5 and 4.0.

### Examples 11-25 and Comparative Examples C-F

The tests of Example 1 were repeated, except that 0.4 g of KBB carbon was used as the catalyst support in place of TiO₂, and the catalyst composition changed as shown in Table 2.

TABLE 2

| Example | Wt % Ru | Wt % Mo | STY | Selectivity |
|---|---|---|---|---|
| Comparative Ex. C | 0.83 | 0.00 | 6.0 | 0.73 |
| Comparative Ex. D | 0.83 | 0.00 | 9.9 | 0.65 |
| Example 11 | 0.83 | 0.17 | 16.1 | 0.81 |
| Example 12 | 0.83 | 0.33 | 16.2 | 0.82 |
| Example 13 | 0.83 | 0.83 | 10.2 | 0.81 |
| Example 14 | 0.83 | 1.33 | 14.0 | 0.83 |
| Comparative Ex. E | 1.65 | 0.00 | 8.3 | 0.65 |
| Comparative Ex. F | 2.48 | 0.00 | 11.5 | 0.63 |
| Comparative Ex. G | 2.48 | 0.00 | 12.5 | 0.60 |
| Comparative Ex. H | 2.48 | 0.00 | 15.6 | 0.58 |
| Comparative Ex. I | 2.48 | 0.00 | 18.6 | 0.69 |
| Example 15 | 2.48 | 0.33 | 20.7 | 0.62 |
| Example 16 a | 2.48 | 0.83 | 29.8 | 0.71 |
| Example 16 b | 2.48 | 0.83 | 35.7 | 0.71 |
| Example 17 | 2.48 | 1.33 | 31.1 | 0.80 |
| Example 18 | 2.48 | 1.67 | 24.5 | 0.79 |
| Comparative Ex. J | 4.13 | 0.00 | 21.4 | 0.54 |
| Example 19 | 4.13 | 0.33 | 29.2 | 0.63 |
| Example 20 | 4.13 | 0.83 | 44.1 | 0.73 |
| Example 21 a | 4.13 | 1.33 | 48.8 | 0.75 |
| Example 21 b | 4.13 | 1.33 | 54.9 | 0.77 |
| Example 22 | 4.13 | 1.67 | 49.9 | 0.78 |
| Example 23 | 5.78 | 1.67 | 45.2 | 0.76 |
| Example 24 | 5.78 | 2.00 | 50.1 | 0.79 |
| Example 25 | 5.78 | 2.33 | 45.3 | 0.79 |

The results above show that Mo increases the activity and selectivity of the Ru catalyst on a carbon support, and that the optimum Ru/Mo weight ratio ranges between 2.5 and 4.0.

### Comparative Examples with Re

The tests of Example 1 were repeated, except that Re₂O₇ was added to the comparative examples in the amounts shown in place of MoO₃ in order to compare the performance of Ru—Re and Ru—Mo. TiO₂ was used as catalyst support. Results are given in Table 3.

TABLE 3

| | Wt % | | | | |
|---|---|---|---|---|---|
| Example | Ru | Wt % Re | Wt % Mo | STY | Selectivity |
| Example 1 a | 2.50 | 0.00 | 0.83 | 63.6 | 0.56 |
| Example 1 b | 2.50 | 0.00 | 0.83 | 35.7 | 0.61 |
| Example 1 c | 2.50 | 0.00 | 0.83 | 58.1 | 0.64 |
| Example 2 | 2.50 | 0.00 | 1.67 | 36.6 | 0.77 |
| Comparative Ex. J | 2.50 | 0.77 | 0.00 | 28.9 | 0.53 |
| Comparative Ex. K | 2.50 | 1.54 | 0.00 | 13.6 | 0.57 |

The above results indicate that the Ru—Mo catalyst on a TiO₂ support is more active and selective than the Ru—Re catalyst for similar weight % loadings.

### Examples 26-28

The tests of Example 1 were repeated, except that SnC₂O₄ was added to Examples 26-28 in addition to the amounts shown of Ru and Mo. Results are given in Table 4.

TABLE 4

| Example | Wt % Ru | Wt % Sn | Wt % Mo | STY | Selectivity |
|---|---|---|---|---|---|
| Example 8 | 5.00 | 0.00 | 1.67 | 32.1 | 0.70 |
| Example 26 | 5.00 | 0.36 | 1.67 | 30.8 | 0.79 |
| Example 27 | 5.00 | 0.50 | 1.67 | 27.3 | 0.84 |
| Example 28 | 5.00 | 0.72 | 1.67 | 18.0 | 0.85 |

The results above indicate that the addition of Sn to the Ru—Mo catalyst on a TiO₂ support increases selectivity.

### Examples 29-34

The tests of Example 1 were repeated, except that SnC₂O₄ was added to Examples 29-34 in addition to the amounts shown of Ru and Mo., and that 0.4 g of KBB carbon was used as catalyst support in place of TiO₂. Results are given in Table 5.

TABLE 5

| Example | Wt % Ru | Wt % Sn | Wt % Mo | STY | Selectivity |
|---|---|---|---|---|---|
| Example 21 a | 4.13 | 0.00 | 1.33 | 48.8 | 0.75 |
| Example 21 b | 4.13 | 0.00 | 1.33 | 54.9 | 0.77 |
| Example 29 | 4.13 | 0.29 | 1.33 | 46.9 | 0.83 |
| Example 30 | 4.13 | 0.57 | 1.33 | 46.7 | 0.87 |
| Example 31 a | 4.13 | 0.86 | 1.33 | 28.3 | 0.88 |
| Example 31 b | 4.13 | 0.86 | 1.33 | 30.0 | 0.87 |
| Example 32 a | 4.13 | 1.15 | 1.33 | 18.9 | 0.88 |
| Example 32 b | 4.13 | 1.15 | 1.33 | 21.9 | 0.87 |
| Example 33 | 4.13 | 1.44 | 1.33 | 22.6 | 0.89 |
| Example 34 | 4.13 | 1.72 | 1.33 | 18.2 | 0.89 |

The results above indicate that the addition of Sn to the Ru—Mo catalyst on a carbon support increases selectivity.

I claim:

1. A hydrogenation catalyst comprising about 0.5% to 15% of ruthenium, about 0.1% to 5% molybdenum and an inert catalyst support, where the percentages are relative to the total weight of support and catalyst, and where the weight ratio of ruthenium to molybdenum is between 2.5 and 4.0.

2. The hydrogenation catalyst of claim 1, comprising about 0.1% to 4% tin.

3. The hydrogenation catalyst of claim 1, comprising about 0.8% to 6% of ruthenium and about 0.1% to 2.5% of molybdenum.

4. The hydrogenation catalyst of claim 3, comprising about 0.1% to 2.0% Sn

5. The hydrogenation catalyst of claim 1, wherein the catalyst support is selected from the group consisting of carbon and titanium dioxide.

6. A method for making tetrahydrofuran, 1,4-butanediol or mixtures thereof by hydrogenating a hydrogenatable precursor in a reactor in the presence of a hydrogenation catalyst comprising about 0.5% to 15% of ruthenium, about 0.1% to 5% molybdenum, and an inert catalyst support, where the percentages are relative to the total weight of support and catalyst, and where the weight ratio of ruthenium to molybdenum is between 2.5 and 4.0. and
recovering at least one hydrogenatable product form the reactor.

7. The method of claim 6, wherein the temperature for the hydrogenation is from 150 to 260° C.

8. The method of claim 6, wherein the hydrogenatable precursor is selected from the group consisting of maleic acid, maleic anhydride, fumaric acid, succinic acid, the esters corresponding to these acids, gamma butyrolactone and mixtures thereof.

9. The method of claim 6, wherein the hydrogenation catalyst comprises about 0.1% to 4% of Sn.

10. The method of claim 6, wherein 1,4-butanediol is predominantly produced at a temperature of 150 to 225° C. and the 1,4-butanediol product is removed from the reactor as a liquid.

11. The method of claim 6, wherein tetrahydrofuran is predominantly produced at a temperature of 225 to 260° C. and the tetrahydrofuran product is removed from the reactor as a vapor.
